(19) United States
(12) Patent Application Publication
(10) Pub. No.: US 2013/0218075 A1
(43) Pub. Date: Aug. 22, 2013

(54) DEVICE FOR IRRADIATING AN INTERNAL BODY SURFACE

(71) Applicant: Yissum Research Development Company of the Hebrew University of Jerusalem Ltd., (US)

(72) Inventors: David Gertz, Jerusalem (IL); Lilach Gavish, Mevasseret Zion (IL)

(73) Assignee: Yissum Research Development Company of the Hebrew University of Jerusalem, Ltd., Jerusalem (IL)

(21) Appl. No.: 13/836,273

(22) Filed: Mar. 15, 2013

Related U.S. Application Data
(63) Continuation-in-part of application No. 13/127,382, filed on Dec. 21, 2011.

(51) Int. Cl. A61N 5/06

(52) U.S. Cl. CPC A61N 5/0601
USPC 604/93.01; 607/92

(57) ABSTRACT

An implantable apparatus for internal treatment of body cavities and damaged vessels using electromagnetic radiation having an implantable light source is operationally connected to an implantable illuminating surface. The implantable light source and implantable illuminating surface are of materials, which are compatible with implantation in a human subject, and possessed of an expanded, deployed configuration and compact, undeployed configuration suitable for laparoscopic insertion within a body cavity for application, when deployed, to an outer, periadventitial surface of an internal vessel or surface of an organ. The apparatus also has a control unit, which control unit regulates the activation of said light source and is optionally located remotely from the implantable light source and illuminating surface.

### CROSS-REFERENCES TO RELATED APPLICATIONS

This application is a continuation-in-part of U.S. patent application Ser. No. 13/127,382, filed Dec. 21, 2011, which is a National Stage of International Application No. PCT/US2009/63154, filed Nov. 3, 2009, which claims the benefit of U.S. Provisional Application No. 61/110,659, filed Nov. 3, 2008, the disclosures of which are incorporated herein by reference.

### BACKGROUND OF THE INVENTION

This invention relates to medical devices and more specifically to such devices for internal irradiation of the body. Application of light to a tissue surface has been used in several medical treatments. Light of certain frequencies and powers has been used to heal and/or modify a variety of pathological states such as opening and/or preventing stenoses in blood vessels, welding of tissues such as torn blood vessel walls or to perform an anastomosis of two blood vessels. Low level laser irradiation appears to prevent the development and retard the progression of vascular aneurysms by bio-stimulating the vessel wall to produce extracellular matrix and reduce inflammation.

Light has also been used for tissue regeneration and therapy. For example, low level laser irradiation in the visible to far-red range of the light spectrum has been shown clinically to accelerate wound healing and reduce pain and inflammation in a variety of immune-based and other musculoskeletal disorders.

The underlying mechanisms of acceleration of wound healing include increasing proliferation (e.g. smooth muscle cells), increasing extracellular matrix protein (e.g. collagen) synthesis, and reduction and or modification of expression and secretion of inflammatory markers. Gavish et al., Lasers in Surgery and Medicine (2006) 38:779-786, which is incorporated herein by reference, discloses that low level laser in vitro stimulates vascular smooth muscle cell proliferation and collagen synthesis, modulates the equilibrium between regulatory matrix remodeling enzymes, and inhibits pro-inflammatory IL-1-β gene expression. In addition, Gavish et al., Lasers in Surgery and Medicine (2008) 40(5): 371-378 which too is incorporated herein by reference, reports that low level laser irradiation of stimulated macrophages inhibits gene expression of monocyte chemotactic protein (MCP-1) as well as the pro-inflammatory cytokines, interleukin (IL)-1a, IL-1b, and IL-6 (and even the anti-inflammatory cytokine IL-10) and downregulates the secretion of pro-inflammatory proteins such as MCP-1 and IL1-beta. These properties of low level laser irradiation would appear to be of profound therapeutic relevance for arterial diseases such as aneurysm where inflammatory processes and weakening of the matrix structure of the arterial wall are the principal underlying pathologic components

An apparatus has been described for applying light to the heart tissue for a biostimulative and cytoprotective effect, which includes a source of electromagnetic radiation and optics operatively connected to the source of electromagnetic radiation.

Additionally, low energy light exposure has been found to both inhibit restenosis following dilation of a stenotic region, and to inhibit vascular spasms, whether or not they are associated with a stenotic region. Such light energy has also been found to arrest progression of a stenosis. An apparatus has been described for exposing the vessel wall to light using a light angioplasty catheter for the prevention of restenosis.

It is also known that an apparatus may be used for applying light to the interior surface of a vascular wall for laser treatment of the vessel. Light may be generated by an extracorporeal source guided by a light guide to the interior of the blood vessel to be treated. A light deflector and diffusor may be used to direct the light in a substantially radial fashion onto the vessel wall.

Abdominal Aortic Aneurysm (AAA) formation is an arteriosclerotic process characterized by marked disruption of the musculo-elastic lamellar structure of the media. Extensive destruction of the wall matrix proteins, including collagen and elastic tissue, is associated with marked inflammatory cell infiltration and progressive diminution in the number of viable smooth muscle cells. Over time, and aggravated by contributory risk factors such as smoking and systolic hypertension, aneurysm growth occurs through a complicated, but insidious, imbalance between matrix protein production and degradation, favoring expansion, thereby increasing the risk of rupture of the weakened wall.

AAA is present in approximately 5 to10% of individuals over the age of 65 years (8% of males over age 65 and 12.5% of males age 75-84), with its frequency increasing as the proportion of elderly individuals in the general population continues to rise. It is widely known that the risk of rupture increases in approximate proportion to aneurysm size, which can be monitored by computed tomography (CT), ultrasound, or magnetic resonance imaging (MRI). The estimated risk of rupture ranges from approximately 10-20% for an abdominal aneurysm 6-7 cm in diameter to 30-50% if the maximum diameter is greater than 8 cm. Overall mortality from a ruptured AAA (outside the hospital) is greater than 90%.

Current forms of aneurysm treatment focus either on the open abdomen, surgical, graft-based repair or endovascular exclusion of the diseased segment of aorta with large, membrane-covered (e.g. Gortex covered) stents. Both techniques have major side effects with potentially life-threatening consequences particularly in patients of advanced age (the majority of patients) or others at high risk or compromised cardiac function.

Increased detection of AAA at early stages of the disease, and the severe complications often associated with currently available surgical and endovascular repair, have emphasized the need for alternative therapeutic strategies that target pathogenetic mechanisms of progression and rupture.

The techniques described in the above references describe generally the benefits of the techniques and methods for using the electromagnetic spectrum to treat tissue surfaces using a remote light source.

### BRIEF SUMMARY OF THE INVENTION

The present invention provides an apparatus and method for the treatment of internally located surfaces of body cavities and damaged internal vessels using electromagnetic irradiation. A device and method according to the invention may be used to radiate an internal tissue surface—for example, but not limited to preventing the progression and rupture of, an arterial aneurysm.

In its first aspect, the present invention provides a device for illuminating a tissue surface. The device is designed for illuminating/irradiating an internal body surface such as, but not limited to an aneurysmatic artery or vein, e.g., an abdominal aortic aneurysm, carotid artery aneurysm, popliteal artery aneurysm, cerebral artery aneurysm, or coronary artery aneurysm. Irradiation of such arterial walls or other organs can be through its perivascular (or periorganal) or endoluminal (or intracavity) surface of an artery or organ.

The device can be powered by an energy source that is located in a variety of positions including, but not limited to: within a body cavity, in a subcutaneous position (e.g. lateral pectoral area similar, but not limited to, the site of positioning of cardiac pacemaker power source/batteries), or in an external position either carried as a purse or any method of transport or positioning.

The device can be connected to its energy source either by immediate juxtaposition or by means of connecting wires which may, in one configuration, pierce the body wall to enter the energy source located in, but not limited to, the subcutaneous position.

Power and duration of the emitted radiation from the device as well as other physical, optical, electrical, and other parameters can be controlled in wireless fashion from outside the body cavity by a portable remote control set.

In one preferred embodiment, the light scatterer of the device is adapted to be implanted inside the body e.g. as a patch with the illuminating surface in its deployed configuration affixed to the body surface to be illuminated. This allows repeated illumination sessions without removing the light scatterer from the body. In one embodiment of the implantable light scatterer, the control unit communicates with the light scattered via a shaft (rigid or flexble) which extends from the light scatterer to the control unit passing through an incision in the skin. The controller may be portable, in which case it could remain attached to the proximal end of the shaft as the user moves around, so that illumination of the body surface could be carried out at any time as required. Alternatively, the control unit may be detachable from the shaft. In this case, the controller would be attached to the shaft at the times when the body surface is to be illuminated. When an implantable light scatterer (patch) is used, the light source may be located in the control unit, in which case light from the light source is conducted to the light scatterer by the light guide. In one embodiment, the light source is implanted together with the illuminating surface. The light source could be connected to the control unit by electrical wires. Alternatively, an energy source for the light source, such as a rechargeable battery, could be implanted together with the light source. In this case, the light source could be provided with a remote control switch for switching the light on and off via wireless communication with the control unit.

This invention provides for an implantable apparatus for internal treatment of body cavities and damaged vessels using electromagnetic radiation or electromagnetic energy said apparatus comprising:
an implantable light source operationally connected to an implantable illuminating surface, which implantable light source and implantable illuminating surface are:
   of materials, which are compatible with implantation in a human subject;
   possessed of an expanded, deployed configuration and compact, undeployed configuration suitable for percutaneous intravascular or laparoscopic insertion within a body cavity for application, when deployed, to an outer, periadventitial surface of an internal vessel or organ; and
a control unit, which control unit regulates the activation of said light source and is optionally located remotely from the implantable light source and illuminating surface.

In some embodiments, such compact undeployed configuration may include a rolled or folded configuration. In some embodiments, such folded configuration contemplates the inclusion of accordion-like pleats/folds in the implantable light source and implantable illuminating surface-containing structure. In some embodiments, such expanded deployed configuration is the unrolled or unfolded version.

In some embodiments, the electromagnetic radiation/energy applied is of wavelengths specifically between 500-900 nanometers.

In some embodiments, implantable light source and implantable illuminating surface are modular, electrically coupled and can be interconnected, whereby the dimensions of each of said implantable light source and implantable illuminating surface may be selected to suit a particular application.

In some embodiments, the modular implantable light source and implantable illuminating surface comprise multiple electrically coupled interconnected modular segments and in some embodiments, such segments can be added or removed at will, and in some embodiments, such choice will reflect a choice for a fit suitable in dimensions for the surface to be irradiated.

In some embodiments, the implantable light source consists of light emitting diode(s) (LED (s), which in some embodiments, is embedded within a matrix comprising the illuminating surface.

In some embodiments, such matrix serves as a supporting structure. In some embodiments, the supporting matrix of the illuminating surface comprises a drug- or other biologic-eluting, or cell-eluting material onto which, or within which, said implantable light source (e.g. LEDs) is/are embedded and operationally connected to said control unit.

In some embodiments, biologics which may be incorporated within the biologic-eluting material include, but are not limited to, matrix metalloproteinase modifiers such as, but not limited to, tetracycline and its derivatives; compounds that interfere with rennin-angiotensin pathways such as, but not limited to, angiotensin converting enzyme inhibitors or angiotensin receptor blockers; drugs of the statin (HMG-CoA reductase inhibitor) family; anti-inflammatory agents (steroidal or non-steroidal); agents that prevent tissue or organ adhesions; antiplatelet or antithrombotic agents; vascular endothelial growth factor and its derivatives, or adult or embryonic, progenitor or mature, cells such as, but not limited to, smooth muscle cell, endothelial cell, or fibroblast lineages.

This invention also provides at least implantable apparatus for internal treatment of body cavities and damaged internal vessels using electromagnetic radiation, comprising: a matrix which matrix is attachable to a tissue surface to be treated. In some embodiments, the invention provides for a plurality of electromagnetic energy sources incorporated on or within said matrix; and a remote electrical source in electrical communication with said plurality of electromagnetic energy sources for activation and control of radiation/energy emitted by said electromagnetic energy sources, wherein said matrix may be in an expanded deployed and compacted (e.g. rolled-up or accordion-folded) undeployed configuration.

In some embodiments, the illuminating device will be implanted and maintained within a body cavity facilitating multiple treatment regimens for the subject. In some embodiments, the illuminating device of the invention may be provided with means for firmly attaching the implanted, deployed illuminating surface to the tissue surface to be treated. Such means may comprise for example small holes at the 4 corners of the illuminating surface for insertion of fine surgical fasteners, clips or sutures for attachment of the illuminating surface to e.g. the peri-adventitial surface of the aorta or to peritoneal surface over the aorta or to any other internal body surface. The skilled artisan will appreciate that any type of fastening, suitable to attach the illuminating surface to the desired body surface may be used.

The light source is selected in accordance with the requirements of the particular application. For example, in order to treat an aneurysm, low level laser irradiation (also known as “low energy laser”, “photo-biostimulation” and “red-light therapy”) in the range of 500 to 900 nm, and more preferably in the range of 600 to 900 nm, may be used that is preferably emitted from the illuminating surface with an energy flux in the range of about 0.01 to about 50 Joules/cm², and more preferably from about 0.1 to about 5 Joules/cm².

In its second aspect, the invention provides a method for treating a tissue surface. In accordance with this aspect of the invention, the method comprises providing an implantable apparatus as herein described onto or adjacent to a tissue surface to be treated, and irradiating the tissue surface.

In some embodiments, the method comprises introducing said implantable apparatus into a body cavity by laparoscopy.

In some embodiments, the method further comprises the step of remote control of electrical power source via, but not limited to, RF coupling.

In some embodiments, the method further comprises varying a radiation wavelength.

In some embodiments, the method further comprises varying a radiation frequency.

In some embodiments, the method further comprises varying a radiation energy level.

In some embodiments, the method further comprises varying a surface treatment by time phasing of the energy from the light source.

The device and method of the invention may be used for illuminating the perivascular surface of a blood vessel, for example, in order to treat an aneurysm. Without wishing to be bound by a particular theory, it is believed that irradiating an aneurysmal blood vessel with low level laser irradiation retards progression of the aneurysm by bio-stimulating the vessel wall to increase smooth muscle cell proliferation, increase extracellular matrix protein production and reduce inflammation.

In one embodiment, the invention provides a device for illuminating a body surface including a shaft having a light guide, a control unit, and a light scatterer. The control unit includes a light source optically coupled to the light guide; and the light scatterer is optically coupled to the light guide. The light scatterer includes a first, rear surface and a second, forward illuminating surface, and the light scatterer has a first undeployed, collapsed configuration and a second deployed configuration in which the illuminating surface has a larger caliber than in the first undeployed, collapsed configuration.

In an alternative embodiment, the invention provides a device for illuminating a body surface which includes an elongated shaft having a light guide, a light source optically coupled to the end of the light guide and illuminating light having a wavelength in the range of 500 to 900 nm, and a light scatterer optically coupled to the end of the light guide.

In another embodiment, the invention also provides a method for illuminating a body surface, which includes providing a device for illuminating a body surface, the device as herein described.

In an alternative embodiment, the invention provides a method for treating an aneurysmal blood vessel including irradiating the blood vessel with radiation having a wavelength from 500 to 900 nm.

In another embodiment, the invention provides an implantable device for irradiating a body surface as herein described. Activation and control of the energy emitted by the light source may be controlled by direct connection of wires, or remotely controlled by an external wireless remote control set. The programmability of the external power source allows for the control and variation of the radiation therapy used, the energy level used, and the radiation frequency at which it is applied without the requirement to move or alter the coverage of the light source.

Such device may be easily constructed, using the stated materials, or other known materials for the assembly and production of the described devices of this invention, for example, by preparing any appropriate biocompatible matrix including matrices that are drug or other biologic material eluting, such as cell eluting, or non-eluting matrices (drug-eluting, cell-eluting, or non-eluting) and applying such matrix to the confines of the illuminating surface such that the LEDs, that are connected by local circuitry one-to-another and to the controller are physically surrounded by such matrix and as such embedded therein, for example by cast molding and other conventional means

Other features and advantages of the present invention will become more apparent from the following detailed description of the preferred embodiments in conjunction with the accompanying drawings, which illustrate, by way of example, the operation of the invention.

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1a and 1b show a device for illuminating a tissue surface in accordance with one embodiment of the invention in deployed and undeployed configurations, respectively

FIGS. 2a and 2b show a device for illuminating a tissue or organ surface or a vascular surface in accordance with a second embodiment of the invention displaying deployed and undeployed configurations.

FIGS. 3a and 3b show a device for illuminating a tissue or organ or vascular surface in accordance with a third embodiment of the invention in deployed and undeployed configurations, respectively.

FIGS. 4a to 4d show use of the device of the invention for treating an aneurysm by laparoscopic procedure. In the implantable embodiment, the illuminating patch/device/swatch (see FIG. 11), can be affixed temporarily or permanently to the periadventitial surface of the aneurysmatic aorta or surface of another organ.

FIG. 5 shows morphometric ultrasonographic measurements of the supra-renal aneurysm prone segment of the subject and the adjacent inter-renal non-aneurysm prone segments of the subject.

FIG. 6 shows a suprarenal abdominal aortic aneurysm 4 weeks after angiotensin II infusion in the apolipoprotein e-deficient mouse (right) not present in similar mouse treated with low level laser irradiation (left).

FIG. 7 shows high-frequency two-dimensional (B-mode) ultrasound measurements of the aortas of control and low level laser irradiated angiotensin II (Ang II) infused apolipoprotein e-deficient mice at baseline and after 4 weeks.

FIG. 8 shows the effect of LLLI on aneurysmal dilatiation of the suprarenal aneurysm-prone aortic segment of angiotensin II-infused apolipoprotein e-deficient mice.

FIG. 9 shows M-mode images of suprarenal aneurysm-prone segments showing marked decrease in radial wall velocity (slope)(RWV) in the severely dilated aorta of the untreated mouse 4 weeks after angiotensin infusion (right) compared to baseline (upper left).

FIG. 10 shows the effect of LLLI on radial wall velocity of the suprarenal Aneurysm Prone Aortic Segment.

FIGS. 11A-C shows an embodiment of an implantable device of this invention.

### DETAILED DESCRIPTION OF THE INVENTION

The invention provides a unique implantable apparatus for internal treatment of body cavities and damaged vessels using electromagnetic radiation or electromagnetic energy. The implantable apparatus of the invention is adaptable to two configurations—deployed and undeployed—which provide advantages during the insertion/implantation of the apparatus within a cavity in a subject. The apparatus also uniquely provides for modular elements enhancing the incorporation of different elements, and different sizes of said elements, by choice. Such unique elements and arrangement of the device provide for more flexible solutions for the subject using the same. The illuminating module of the implantable apparatus can, in some embodiments, be detached from the introducing shaft so as to permit temporary or permanent attachment to the outer or inner surface of the vessel or organ to be illuminated. The multi-solution, implantable illuminating module can comprise LED's (emitting any of a variety of light frequencies) as the illuminating elements, and can contain a drug-eluting, chemical-eluting, and/or cell-eluting matrix into which the LED's are embedded,

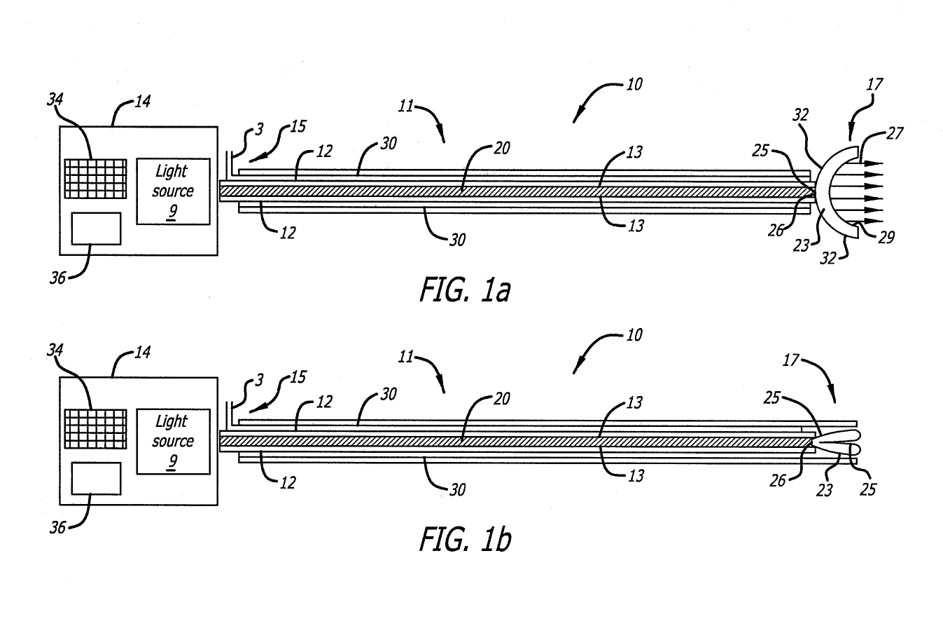

The present invention shows a device in FIG. 1 generally indicated by 10, in deployed and undeployed configurations, for illuminating a tissue surface to be treated, in accordance with one embodiment of the invention. The tissue surface may be, for example, the outer surface of a blood vessel where an aneurysm has formed. The illuminating device has a slender shaft 11, shown in longitudinal section in FIG. 1, having a proximal end 15 and a distal end 17. The shaft 11 may be rigid or flexible, as required in any application. The shaft 11 has a sheath 13 surrounding a light guide 20 that may consist of a single optical fiber or a bundle of optical fibers.

The shaft 11 is connected at its proximal end 15 to a control unit 14 that houses a light source 9, that may be, for example, a laser. Light generated by the light source 9, enters the light guide 20 and is conducted through the light guide 20 to the distal end of the light guide 20. Since the end face 26 of the distal end of the light guide 20 is flat, the pencil of light emerging from the end face 26 will have a cross-sectional area essentially equal to the cross-sectional area of the light guide 20. Thus, in accordance with the invention, in order to increase the radiated area, the radiation device 10 further comprises a light scatterer 23 positioned at the distal end of the light guide 20, that is optically coupled to the light guide 20. The light scatterer 23 has a deployed configuration shown in FIG. 1a in which an illuminating surface 29 has a large caliber configuration. Light emitted from the end face 26 of the light guide 20 enters the light scatterer 23 at a first surface 25 and is scattered through the light scatterer 23. The light is then emitted from the illuminating surface 29, as indicated by the arrows 27 to radiate the site to be treated, as described below. The illuminating surface has an area that is greater than the cross-sectional area of the light guide 20. In the deployed configuration, the illuminating surface 29 is preferably shaped to conform to the surface to be radiated so that the illuminating surface can be applied onto the surface to be radiated. For example, for illuminating the outer surface of a blood vessel, the illuminating surface 29 would be a partial cylindrical surface, as shown in FIG. 1a. As explained below, this enhances coupling of the light radiated from the illuminating surface and radiation of the surface to be treated. The light scatterer is preferably provided with a light reflecting coating 32 on its rear surface in order to reflect back scattered light in the light scatterer in the direction of the arrows 27.

The light scatterer 23 also has an undeployed configuration shown in FIG. 1b in which the illuminating surface 29 is collapsed into a small caliber configuration. In the embodiment shown in FIG. 1, the light scatterer 23 is formed from a resiliently flexible material. The light scatterer may be made, for example, from transparent silicon rubber in which a light scattering substance is embedded. Alternatively, the light scatterer may include one or more lenses (not shown). In this embodiment, the shaft 11 includes a constraining sleeve 30 that surrounds the sheath 13. In the undeployed configuration shown in FIG. 1b, the light scatterer 23 is constricted into its small caliber undeployed configuration and is maintained in the undeployed configuration by means of the constraining sleeve 30. The constraining sleeve 30 is slidable axially along the shaft 11 from a forward position shown in FIG. 1b and a rearward position shown in FIG. 1a. In the forward position (FIG. 1b), the sleeve 30 extends beyond the end of the optic fiber 20 with the light scatterer 23 collapsed in the interior of the sleeve 30. When the sleeve 30 is brought to its rearward position (FIG. 1a) the sleeve 30 is retracted from the light scatterer 30 and the light scatterer 23 spontaneously assumes its deployed, large caliber configuration due to the resiliently flexible character of the light scatterer 23. In order to slide the sleeve 30 between its forward and rearward position, a user may grasp the sleeve 30 at its proximal end and manually slide the sleeve over the sheath 13.

The illuminating device 10 may further be configured for connection to a source of negative pressure. As shown in FIG. 1, for this purpose, the shaft may include a channel 12 extending from a valve 3 adapted for connection to a source of negative pressure (not shown) at the proximal end of the shaft 11 through the shaft 11 to the distal end of the shaft. As explained below, generation of negative pressure at the distal end of the light scatterer 23 is used to attach the light scatterer 23 to the tissue surface and to immobilize the light scatterer on the tissue surface during radiation.

The shaft 11 may optionally contain a working channel (not shown) in order to accommodate a guide wire or working tool, as required in any application.

The control unit 14 is provided with a user input device, such as a keypad 34 to allow the user to select one or more parameters of the treatment, such as the radiation intensity or fluency. The control unit may also have a display 36 such as a screen 38 displaying the selected parameters and other relevant information.

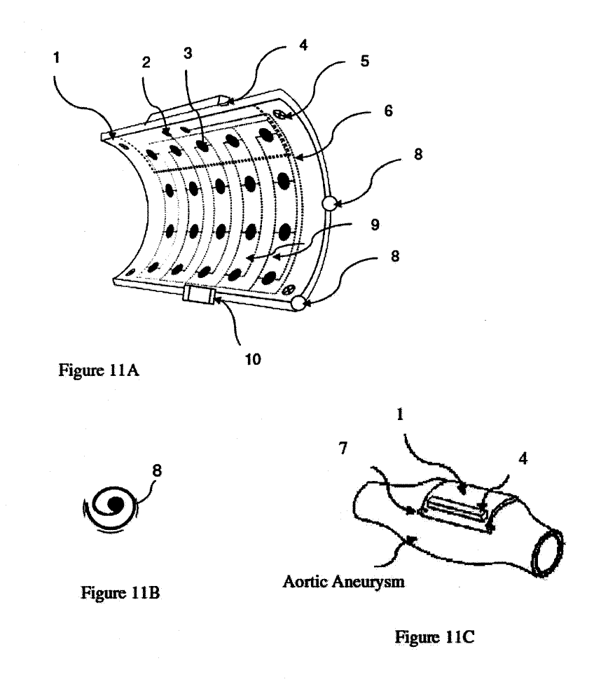

In one preferred embodiment, the light scatterer 23 of the device is adapted to be separated from the introducing shaft and implanted inside the body with the illuminating surface 29 in its deployed configuration and affixed to the body surface to be illuminated. FIG. 11 provides a more detailed view of an embodied implantable device of this invention. Such a device allows repeated illumination sessions without removing the light source from the body. In one embodiment of the implantable light source, the control unit 14 communicates with the light scatterer 23 via the shaft 11 which extends from the light scatterer 23 to the control unit 14, passing through an incision in the skin. The controller may be portable, in which case it could remain attached to the proximal end of the shaft as the user moves around, so that illumination of the body surface could be carried out at any time as required. Alternatively, the control unit may be detachable from the shaft. In this case, the controller would be attached to the shaft at the times when the body surface is to be illuminated. When an implantable light scatterer is used, the light source may be located in the control unit, in which case light from the light source is conducted to the light scatterer by the light guide 20. In one embodiment, the light source is implanted together with the illuminating surface. The light source could be connected to the control unit by electrical wires. Alternatively, an energy source for the light source, such as a rechargeable battery, could be implanted together with the light source. In this case, the light source could be provided with a remote control switch for switching of the light on and off that is controlled via wireless communication with the control unit.

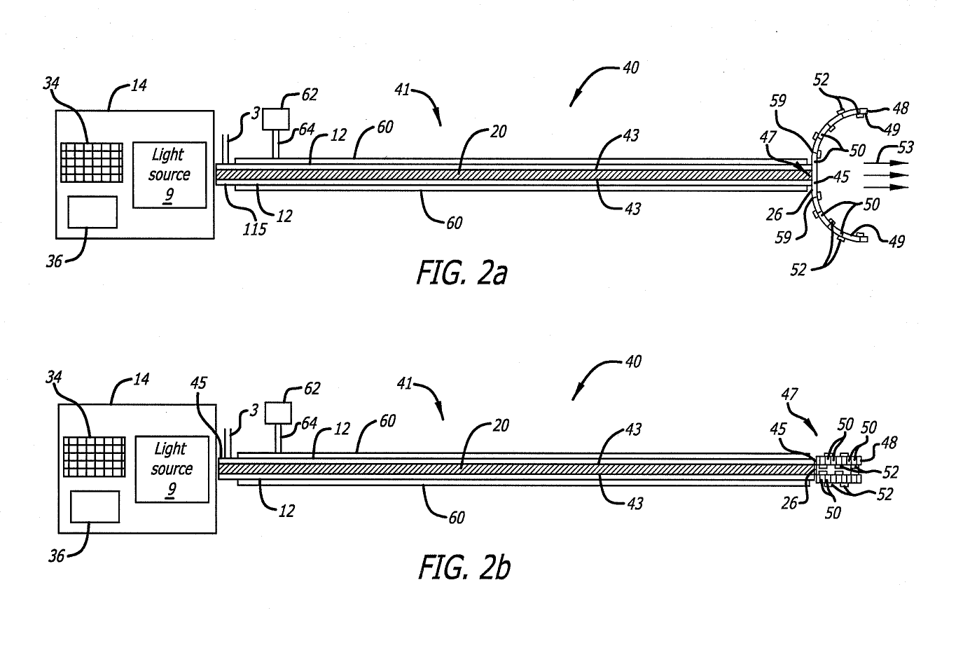

FIGS. 2a and 2b show a device, generally indicated by 40, (in deployed and undeployed configurations respectively) for illuminating a tissue surface to be treated, in accordance with another embodiment of the invention. The tissue illuminating device 40 has several components in common with the device 10 described above in reference to FIG. 1, and similar components are indicated by the same reference numerals in FIGS. 1a and 1b and 2a and 2b without further comment. The illuminating device has a slender shaft 41, shown in longitudinal section in FIG. 2, having a proximal end 45 and a distal end 47. The shaft 41 may be rigid or flexible, as required in any application. The shaft 41 has a sheath 43 surrounding a light guide 20 that may consist of a single optical fiber or a bundle of optical fibers.

The device 40 includes a light scatterer 48 at the distal end 47 of the shaft that is optically coupled to the light guide 20. The light scatterer 48 has a large caliber deployed configuration shown in FIG. 2a, and a small caliber undeployed configuration shown in FIG. 2b. In this embodiment, the light scatterer 48 includes a pleated sheet containing two or more panels 50 that are hinged together by hinges 52. In the undeployed configuration (FIG. 2b) the pleated sheet is folded into the small caliber, while in the deployed configuration (FIG. 2a) the pleated sheet is extended. The panels may be formed from transparent silicone rubber with or without any other polymeric matrix.(eluting or non-eluting as described herein) in which a light scattering substance is embedded Light emitted from the end face 26 of the light guide 20 enters the light scatterer 48 at a first surface 45 and is scattered through the light scatterer 48. The light is then emitted from an illuminating surface 49 on each panel in an essentially forward direction, as indicated by the arrows 53 to radiate the site to be treated. In the deployed configuration, the illuminating surface 49 is preferably shaped to conform to the surface to be radiated so that the illuminating surface can be applied onto the surface to be radiated. The light scatterer is preferably provided with a light reflecting coating 59 on its rear surface in order to reflect back scattered light in the light scatterer in the direction of the arrows 53. The reflecting coating 59 may be made, from a biocompatible shiny material, deposited on the rear surface of the light scatterer.

The light scatterer 48 further includes an actuating mechanism for transforming the light scatterer 48 between its deployed and undeployed configurations. The hinges 52 comprise one or more elements faulted from a shape memory material such as Nitinol that has been trained to behave as described below. The hinges have a deployed configuration shown in FIG. 2a, and an undeployed configuration shown in FIG. 2b. The hinges are attached to the panels so that passage of the elements from their undeployed to their deployed configurations drives the passage of the light scatterer 48 between its undeployed configuration and its deployed configuration, and vice versa.

The shaft 41 has a channel 60 for delivering a pressurized liquid such as physiological saline from a fluid source 62 located adjacent to, or inside, the control unit 14. The fluid source 62 includes a temperature controlling system that allows the temperature of the fluid to be selected by a user. The fluid source 62 is in fluid contact with the channel 60 via a connecting hose 64. When the light scatterer 48 in its undeployed configuration is to be brought to its deployed configuration, a pressurized fluid is used at a first temperature. The fluid is delivered to the distal end 47 of the shaft where it brings the temperature of the hinge elements to a temperature at which the shape memory material undergoes a first shape transition bringing the hinges 52 into their deployed configuration. When the light scatterer 48 in its deployed configuration is to be brought to its undeployed configuration, a pressurized fluid is used at a second temperature that is delivered to the distal end of the shaft where it brings the temperature of the hinges 52 to a temperature at which the shape memory material undergoes a second shape transition bringing the hinges 52 into their undeployed configuration. Alternatively in the undeployed configuration the implantable scatterer may exist in a rolled-up form and then converted to the deployed by unrolling. Unrolling may be performed by mechanical manipulation with the aid of appropriate surgical instruments (e.g. laparoscopic instruments/tools.

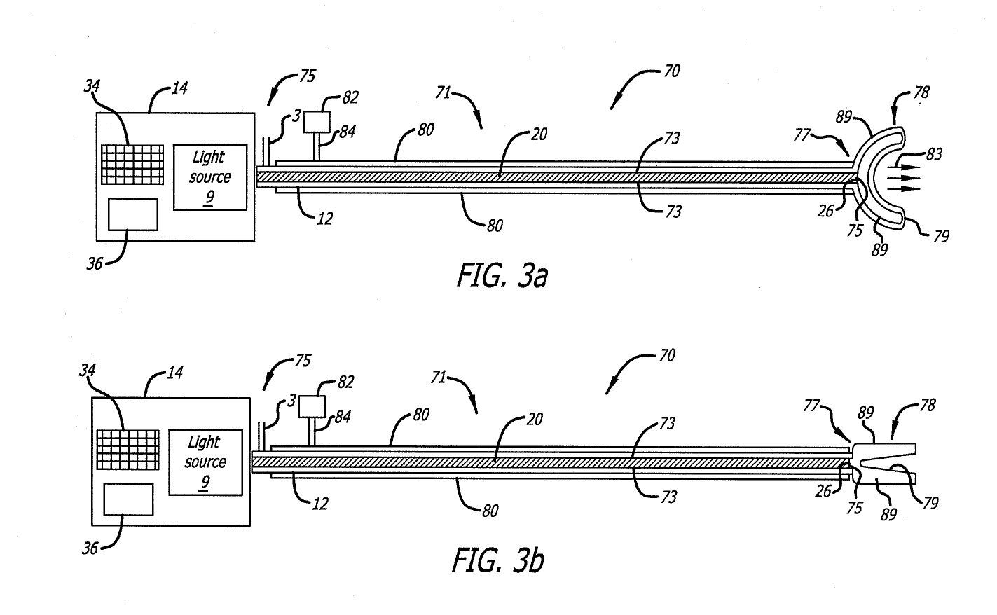

FIGS. 3a and 3b shows a device, generally indicated by 70, for illuminating a tissue surface to be treated, in accordance with yet another embodiment of the invention. The tissue illuminating device 70 has several components in common with the device 10 described above in reference to FIGS. 1a and 1b, and similar components are indicated by the same reference numerals in FIGS. 1a, 1b, 3a, and 3b, without further comment. The illuminating device has a slender shaft 71, shown in longitudinal section in FIG. 3, having a proximal end 75 and a distal end 77. The shaft 71 may be rigid or flexible, as required in any application. The shaft 71 has a sheath 73 surrounding a light guide 20 that may consist of a single optical fiber or a bundle of optical fibers.

The device 70 includes a light scatterer 78 at the distal 77 of the shaft that is optically coupled to the light guide 20. In this embodiment, the light scatterer 78 is an inflatable balloon that may be formed, for example, from transparent silicone rubber with or without any other polymeric material (as described in other parts of the current application) in which a light scattering substance is embedded. The light scatterer 78 has a large caliber deployed configuration shown in FIGS. 3a and 3b in which the balloon is inflated, and a small caliber undeployed configuration shown in FIG. 3b in which the balloon is deflated. In the deployed configuration, light emitted from the end face 26 of the light guide 20 enters the light scatterer 78 at a first surface 75 and is scattered through the light scatterer 78. The light is then emitted from an illuminating surface 79 of the light scatterer 78 in an essentially forward direction, as indicated by the arrows 83 to radiate the site to be treated. In the deployed configuration, the illuminating surface 79 is preferably shaped to conform to the surface to be irradiated. The light scatterer is preferably provided with a light reflecting coating 89 on its rear surface in order to reflect back scattered light in the light scatterer in the direction of the arrows 83.

The shaft 71 has, a channel 80 for delivering a pressurized fluid such as water or air from a fluid source 82 located adjacent to, or inside, the control unit 14. The fluid source 82 is in fluid contact with the channel 80 via a connecting hose 84. When the light scatterer 78 in its undeployed configuration is to be brought to its deployed configuration, the pressurized fluid is delivered to the distal end 47 of the shaft and inflates the balloon.

When the light scatterer 78 in its deployed configuration is to be brought to its undeployed configuration, the fluid is pumped from the balloon back to the fluid source 82.

In some embodiments, conversion from undeployed to deployed configuration may also be accomplished by simple mechanical manipulation by rotating a knob located on the insertion shaft that is connected to detachable wires connected to two parallel edges of the of the scatterer. Deployment is typically performed within the body cavity prior to fixation of the scatterer to the surface to be illuminated. The apparatus is also fitted with a release button to detach the implantable scatterer from the shaft. Such release button may be located on said rotating knob or elsewhere along the introducing shaft. After deployment, and either before or after detachment from the shaft, the scatterer can then be fixed to the surface to be illuminated by surgical clips, fasteners, or sutures connected to the corners of the scatterer.

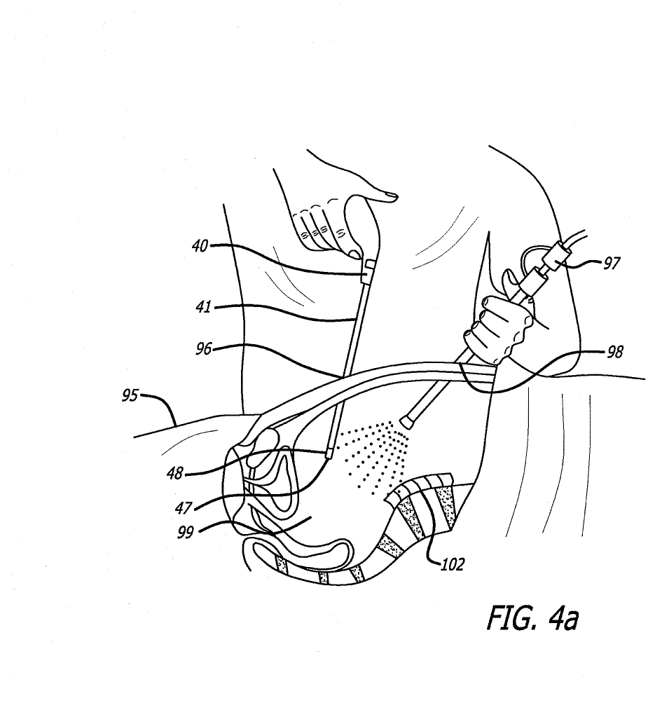

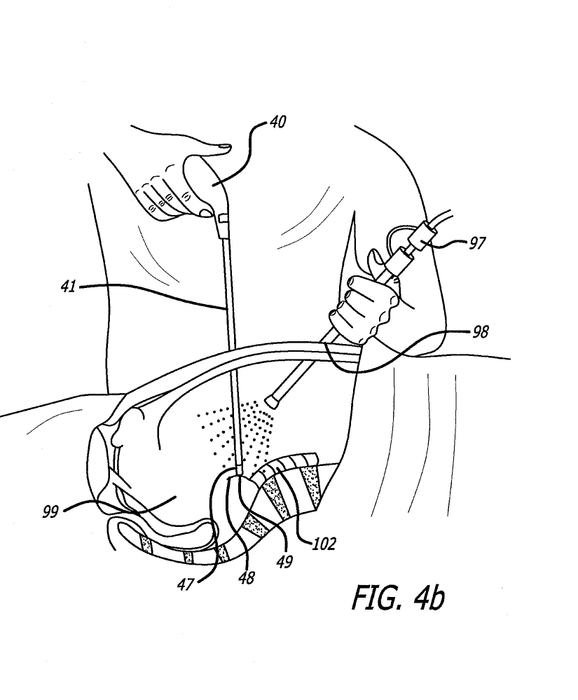

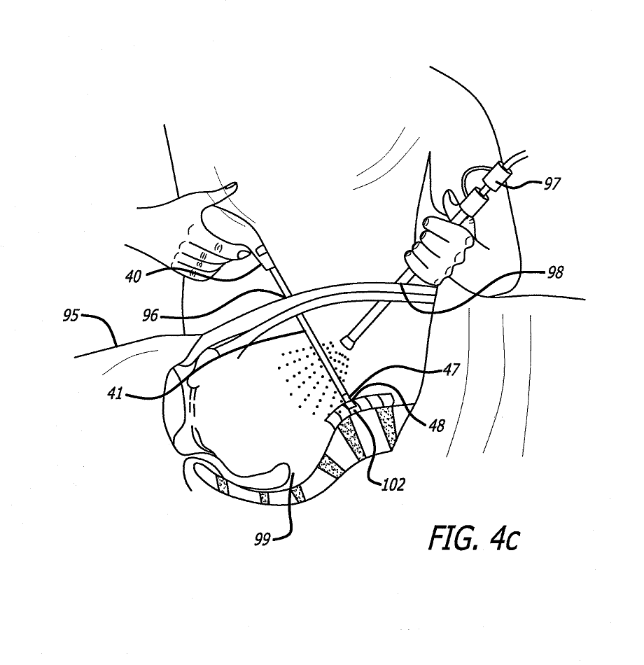

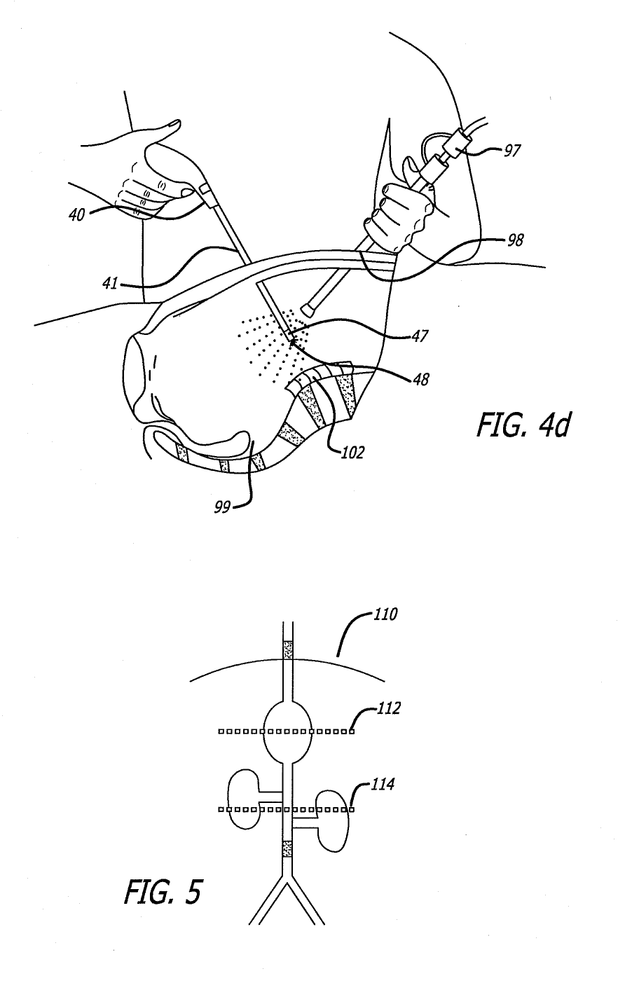

FIGS. 4a and 4b depict use of the device 40 in a surgical procedure in which an internal body surface is to be radiated. In the example, the surgical procedure is treatment of an aneurysm in the abdominal aorta 102. This is by way of example only, and the device of the invention may be used to radiate any body surface. As shown in FIGS. 4a and 4b, the shaft 41 of the device 40, with the light scatterer 48 in its undeployed configuration, is introduced through an incision at a first location 96 on the body surface of a subject 95 into a body cavity, which in this example, is an abdomen 99. The surgical procedure may utilize laparoscopy, in which case an endoscope 97 is introduced into the abdomen 99 through a second incision at a second location 98 on the body surface. Abdominal body organs (not shown in FIGS. 4a-4d) are moved aside in order to allow access to the aorta 102. The endoscope 97 illuminates the outer (periadventitial) surface of the aorta 102 or any other organ within the abdomen 99. The endoscope 97 is part of a laparoscopic imaging system that displays on a display screen (not shown), an image of the contents of the abdomen 99, so as to allow a user 110 to observe the cavity 99 during the procedure. The abdominal cavity 99 may be temporarily inflated in order to facilitate the maneuverability of the device 40 and the endoscope 97 in the abdomen 99.

In some embodiments, the light scatterer may be detached from the shaft and implanted as a patch onto the surface of the aorta or other organ or surface for irradiation of same (FIG. 11).

In FIG. 4a, the device 40 has been maneuvered so as to bring the distal end 47 of the shaft 41 and the light scatterer 48 into proximity with the aorta 102. At this point, the fluid in the fluid source 62 (FIG. 2) is brought to the first temperature, and the fluid source 62 is then activated in order to deliver the fluid at the first temperature through the connecting hose 64 (FIGS. 2a and 2b) and the channel 60 (FIGS. 2a and 2b) to the distal end 47 of the shaft where it brings the hinge elements to a temperature in which they assume their deployed configuration. This brings the light scatterer 48 to its deployed configuration, as shown in FIG. 4b. The illuminating surface 49 of the light scatterer 48 in the deployed configuration of the light scatterer 48 has the shape of a partial cylindrical surface with a radius approximately equal to the outer radius of the aorta 102 to be radiated. The device 40 is then maneuvered in the abdomen 99 so as to apply the illuminating surface 49 to the outer surface of the aorta 102, as shown in FIG. 4c. The valve 3 is then opened to deliver negative pressure to the light scatterer 48 so as to firmly apply the illuminating surface 49 to the aorta and to immobilize the light scatterer 48 on the outer surface of the aorta. The light source in the control unit 14 is then activated. Light from the light source is conducted along the light guide 20 to the light scatterer 48. Essentially the entire surface area of the aorta that is in contact with the illuminating surface 49 is simultaneously radiated. Alternatively, the implantable patch may be brought to its deployed configuration and applied to the surface to be illuminated by manipulation with surgical instruments compatible with laparoscopic equipment and procedures, and then detached and withdrawn from said surface when radiation is completed.

When the radiation is completed, the negative pressure is discontinued to release the light scatterer 48 from the aorta. The fluid in the fluid source 62 is brought to the second temperature, and the fluid source 62 is then activated in order to deliver the fluid at the second temperature through the connecting hose 64 and the channel 60 to the distal end 47 of the shaft where it brings the hinge elements to a temperature in which they assume their undeployed configuration. This brings the light scatterer 48 back to its undeployed configuration, as shown in FIG. 4d. The device 40 is then removed from the abdomen 99 See also alternative listed in 0074 above.

Implantable devices that provide electromagnetic irradiation of internal tissues, organs, or organ surfaces or, alternatively, direct illumination of the blood from the interior of a blood vessel have been described; however, such devices are either in tubular or flat in form. The implantable device described in the current application is suitable for illumination of the wall of the blood vessel, or other organ, from its exterior (periadventitial) surface or from its internal (luminal) surface. The device has in one and the same instrument, both deployed and non-deployed configurations the latter serving the purpose of miniaturization (rolled or accordion-like folded) for endoscopic or percutaneous intraluminal or intracavity or laparoscopic passage into a body cavity (see FIGS. 2a and 2b and FIG. 11). The device allows for electromagnetic irradiation (including, but not limited to, low power, low thermal radiation in the visible to the far red range [specifically but not limited to 500 to 900 nanometers]) not only of the interior of a blood vessel or other body cavity in its complete, unseparated undetached form (in one embodiment); but, this device is also configured (in a second embodiment) for detachment of the illuminating (light scatterer) surface and implantation of the latter as, for example, a patch on to the exterior (periadventitial) surface of the blood vessel (or other body cavity). (note: perivascular implantation minimizes mechanical damage to the delicate interior (e.g. endothelial) surface of the blood vessel or other cavity and reduces the likelihood of life-threatening thrombosis). Moreover, the current device is configured in a modular fashion, built of multiple electrically coupled interconnected modular segments that can be added or removed at will in accordance with the dimensions of the surface to be irradiated such as an aneurysm.

In some embodiments, the device of this invention is configured for implantation within the abdominal cavity (or any other body cavity) by laparoscopy, and, as such, is structured to interface, and be compatible, with all conventional laparoscopic or other endoscopic catheters and equipment.

FIG. 11 depicts an implantable, modular scatterer unit/patch/swatch=1 that can be applied to the periadventitial surface of a vessesl (or surface of another organ). In this embodiment the implantable patch consists of a structural matrix into which are embedded LEDs and circuitry connected to an energy source that enables activation and control. The said matrix can be a drug, chemical, or cell eluting matrix. Circuitry between contol unit and LEDs=2; LEDs=3; Energy source, control unit and processer=4;

In one preferred embodiment, such a device allows repeated illumination sessions without removing the light source from the body. In one embodiment the control unit 4 communicates with the LEDs 3. The controller may remain attached to the device, or alternatively, the control unit may be remotely located. The LEDs may, in some embodiments, be connected to the control unit by electrical wires 6,3. Alternatively, an energy source for the light source, such as a rechargeable battery, could be implanted together with the light source. In this case, the light source could be provided with a remote control switch for switching of the light on and off that is controlled via wireless communication with the control unit.

There may be included, in some embodiments, a region for promoting fixation to a body surface 5; for example, by including holes or voids, through which or to which a clip, fastener or suture may be applied. An example of the incorporation of a fixation element 7, such as a fastener, clip or suture placed there through is shown. Regions for the application/insertion of a shaft, such as connection ports 8, are shown, as well. A structural matrix into which light elements are embedded 9 are shown, and such matrix may be a drug-eluting or cell-eluting matrix. In some aspects, there may be included a connection port 10, which may be useful in providing for electrical coupling of additional modules to enlarge the area of the implantable light source, for example, in order to expand the dimensions of the illuminating surface to be appropriate for a larger surface to be irradiated. Such connection ports may be along any edge or surface, to provide enhanced flexibility of design to suit a particular application.

Comparing FIGS. 11A versus 11B provides an embodiment of a deployed versus undeployed configuration (FIG. 11A versus 11B, respectively). In the depicted embodiment, the implantable light source and implantable illuminating surface are rolled up in the undeployed configuration connected to the shaft and within a cylindrical protector. Such cylindrical protector can be removed or withdrawn prior to, or upon, deployment, as in other embodiments, and the patch “unrolled” for implantation. It will be apparent to the skilled artisan, however, that other undeployed compacted configurations are feasible, including folded configurations, and including accordion folded configuations, and others.

Such compacted configurations provide for a more reliable insertion within the subject particularly suitable for insertion procedures, which do not require large incisions or greater internal tissue exposure such as laparoscopy or any other endoscopic procedure.

In some embodiments, a tool may be inserted, for example to facilitate manipulation of the implantable patch, which tool attaches to the implantable light source/illuminating surface, for example, a thin tool containing a terminal hook, which can attach to a region of the implantable light source/illuminating surface when undeployed, and facilitate the extension and deployment of the same.

In some embodiments, when deployed, the implantable light source/illuminating surface is then affixed to a desired site or surface—for example, by attaching a fastener, suturing or gluing the implantable light source and implantable illuminating surface to the desired tissue surface.

In some embodiments, the device further provides a system/means for drug elution, and/or cell elution, for delivery to a body surface such as, but not limited to, a pathologic blood vessel such as, but not limited to, an aneurysmatic abdominal aorta.

In some embodiments, the implantable device transmits light primarily, but not necessarily limited to wavelengths of the electromagenetic spectrum between 500 and 900 nanometers in forms such as, but not limited to, laser or light emitting diodes.

In some embodiments, the implantable scatterer (patch) portion of the device is configured in a modular fashion, built of multiple electrically coupled interconnected modular segments that can be added or removed at will in accordance with the dimensions of the surface to be irradiated such as an aneurysm (FIG. 11).

In some embodiments, the device comprises a drug eluting and/or cell eluting material that permits time- and dose-controlled release of a variety of pharmacological/chemical agents and or cells and or other biologics to the wall of the affected blood vessel or body surface at the same time of, or intermittently with, light therapy.

In some embodiments, the device permits controlled release of pharmacological agents such as, but not limited to matrix metalloproteinase modifiers such as, but not limited to, tetracycline compounds and their analogues and derivatives, anti-inflammatory agents (e.g. steroidal or non-steroidal), antiplatelet or antithrombotic agents, modifiers of the rennin-angiotensin system such as angiotensin converting enzyme inhibitors and angiotensin receptor blockers and their analogues and derivatives, statins (I-IMG-CoA reductase inhibitors) and their analogues and derivatives, agents to prevent adhesions of the affected vessel with adjacent organs such as intestines, other antimicrobial agents to prevent infection, etc.

In some embodiments, the drug, chemical, biologic, or cell eluting material or matrix may include, but is not necessarily limited to, any of a variety of known polymeric, drug-eluting or cell-eluting matrices such as, but not limited to, polylactic, polyglycolic acid compounds and their analogues or derivatives or collagen or other protein matrices and their analogues or derivatives. Possible eluting biologics contained within such matrix may include, but are not limited to, matrix metalloproteinase modifiers such as tetracycline and its analogues and derivatives, compounds that interfere with rennin-angiotensin pathways such as angiotensin converting enzyme inhibitors or angiotensin receptor blockers and their analogues and derivatives, antiplatelet or antithrombotic agents; statins (HMG-CoA reductase inhibitors) and their analogues and derivatives, anti-inflammatory agents (steroidal and non-steroidal) and their analogues or derivatives, compounds that prevent the development of adhesions; vascular endothelial growth factor and its analogues or derivatives, adult or embryonic, progenitor or mature, cells such as, but not limited to, smooth muscle progenitor or adult cells or endothelial progenitor or adult cells, or fibroblasts (adult or progenitor)]

The drug/chemical/cell eluting feature of the implantable device is mediated by a matrix formed from, but not necessarily limited to, any of a variety of known drug eluting and/or releasing films, chemicals, compounds, coatings, or polymers.

In some embodiments, the eluting matrix feature of this implantable, multifunctional device permits transfer of cells to the affected vascular surface such as, but not limited to, human or animal differentiated cells, or human or animal adult or embryonic progenitor cells of, but not limited to, smooth muscle cells or fibroblasts, or any other human or animal adult or progenitor/stem cells, in order to, but not only to, further strengthen the weakened vascular wall. Strengthening the weakened (e.g. aneurysmatic) vascular wall is thereby additionally facilitated by these features of the device by increasing the number of resident cells within the vascular wall including, but not limited to, smooth muscle cells or fibroblasts and increasing the elaboration of matrix proteins known to be produced by these cells such as, but not limited to, several types of collagen and/or other matrix proteins. Based on previous studies by the inventors and others, the light emitting feature of this implantable device would further facilitate the proliferation of the (e.g. smooth muscle, fibroblasts, or other) cells once implanted within the weakened arterial wall and facilitate the appropriate incorporation of these cells within the local microenvironment of the affected artery.

One embodiment of the implantable drug/chemical/cell eluting, light emitting device is a patch for perivascular delivery to the perivascular surface of the aneurysm or body structure. This scatterer/patch can be delivered to the site by a variety of techniques including, but not limited to, directly, by surgical insertion, by laparoscopy, or endovascularly. The implantable device, in its perivascular embodiment, is compatible and interfaceable with all necessary laparoscopic equipment.

Another embodiment of the implantable drug /chemical/cell eluting, light emitting device is a patch for endoluminal, intravascular delivery to the endovascular surface of the aneurysm or body surface. In this endoluminal embodiment, the device is compatible and interfaceable with all necessary intravascular catheterization equipment.

Possible routes of delivery and uses of the device include, but are not limited to:
1. laparoscopic trans-abdominal delivery to the perivascular surface of the aorta affected by, but not limited to an abdominal aortic aneurysm.
2. Endoscopic, transthoracic delivery to the perivascular surface of the ascending aorta or coronary arteries affected by, but not limited to, aneurysm.
3. Endoscopic, transcranial delivery to the perivascular surface of intracranial blood vessels affected by, but not limited to, aneurysm.
4. Endoscopic or other percutaneous delivery to any other vessel such as peripheral arteries such as, but not limited to, popliteal artery aneurysms of the leg.
5. Intravascular, delivery to the endoluminal surface of an affected artery, such as, but not limited to aneurysms of, the abdominal or ascending aorta, cerebral arteries, popliteal arteries, or coronary arteries.
6. Direct application to the skin surface of a relatively superficially located aneurysm such as, but not limited to, popliteal aneurysm.
7. Direct surgical application to an internal body surface.

In some embodiments, the implantable periarterial device can be withdrawn after one treatment or fastened to or clipped by included attached tissue clips or other fasteners, or sutured to the blood vessel, or to tissues adjacent to the blood vessel, for multiple treatments.

In some embodiments, the device is under wireless (including, but not limited to, RF) control permitting multiple treatment without multiple invasions. In one embodiment, the patch incorporates an internally embedded power source. In another embodiment, the device is connected to an external power source.

Biological Experimental Data

Study Design

The effect of low level laser irradiation (LLLI) on AAA formation in the angiotensin II-infused apolipoprotein E-deficient (-/-) C57/Black mouse model was studied. This model was developed in the laboratory of Daugherty and colleagues. In this mouse model, suprarenal abdominal aortic aneurysms form in up to 80-85% of the cases. High frequency ultrasonography (HF-U/S) (0.01 mm resolution) was used in the laboratory in order to quantify the effect of LLLI on aortic expansion over time. This recently developed technology was designed specifically for non-invasive micro-imaging in mice.

Angiotensin II was infused in twenty-eight male mice aged 12-13 weeks via subcutaneously implanted osmotic minipumps (see details below). Laparotomy was performed to enable direct irradiation of the aorta. Nine animals died during surgery, and 1 was disqualified as a result of pump extrusion. Of the 28 mice, 13 were irradiated and 15 were sham-operated, non-irradiated controls.

Referring to FIG. 5, morphometric ultrasonographic measurements generally indicated by 110, of the supra-renal aneurysm prone segment 112 and the adjacent inter-renal non-aneurysm prone segments 114 were performed at baseline and at 4 weeks after the onset of angiotensin II infusion.

Mice

The mice were bred in-house from stock originating from Jackson Laboratories. The mice were housed in a specific pathogen-free (SPF) environment. Water and normal diet were available ad libitum. The investigation conforms with the Guide for the Care and Use of Laboratory Animals published by the US National Institutes of Health (NIH Publication No. 85-23, revised 1996). Animal care and the experimental procedures were approved by the Ethics Committee of the Faculty of Medicine of The Hebrew University, Jerusalem, Israel (MD-07-10349-3).

Angiotensin II Infusion

Osmotic minipumps (Alzet, model 2004, Durect Corp; Cupertino, Calif.) were filled with Angiotensin II (Calbiochem; La Jolla, Calif.) (infusion rate 1000 ng/kg/min). The pumps were implanted subcutaneously on the right flank through an incision in the scapular region and maintained for the entire 28 days—from the time of irradiation until sacrifice.

Low Level Laser Irradiation (LLLI)

A diode laser system coupled to an optic fiber was used with 0-450 mW power and 780 nm wavelength (BWTek, Newark, Del.). The irradiation box contained 2 compartments with a hole between. The laser was placed in the upper compartment with the optic fiber tip threaded through the hole above the irradiation plane at a distance adjusted for optimal expansion of the ray. The power was measured at the plane of the aorta with a Laser Mate power meter (Coherent, Auburn group, Europe). The exposed aorta was irradiated at 4 mW/cm for 9 minutes which accumulated to a total energy density of 2 J/cm.

Surgical Protocol

Mice were anesthetized by subcutaneous injection of ketamine (200 mg/kg) and xylazine (10 mg/kg). All animals received subcutaneous injections of cefamizine (30 mg/kg), Tramadol analgesia (2 mg/kg), and warned saline (2 ml). Chloramphenicol ointment was applied locally to the conjunctival sacs to prevent corneal damage. The abdominal aorta was exposed through a left subcostal incision (retroperitoneal approach), and the region between the diaphragm and the renal arteries was isolated from the surrounding retroperitoneal structures. The mice were then placed in the irradiation box with the exposed abdominal aorta localized in the center of the beam. The sham-operated, non-irradiated control animals followed the same protocol but with the laser turned off. The Alzet minipump was implanted as described above.

Ultrasound Imaging and Analysis

The high resolution ultrasound imaging system Vevo 770, VisualSonics,

Toronto, Canada) was used to perform two-dimensional (B-mode) and motion-mode (M-mode) imaging using a mechanical transducer (RMV707B) synchronized to the electrocardiographic signal. The transducer had a central frequency of 40 MHz, a focal length of 6 mm, a frame rate of 30 Hz, and an 8×8 mm field of view with spatial resolution of 30 μm. Scans were performed under anesthesia using 2% isofluorane. A longitudinal image of the abdominal aorta between the diaphragm and the renal arteries was acquired. Doppler signals were used to confirm the identification of the abdominal aorta. Transverse images at the level of the maximal dilatation of the aneurysm-prone suprarenal portion of the abdominal aorta were acquired in B-mode and M-mode with the adjacent, non-aneurysm-prone portion of the aorta between the right and left renal arteries serving as internal controls (FIG. 1). The measurements were performed with VisualSonics proprietary software using multiple frames. The maximum aortic cross-sectional diameter (associated with systole) was determined from B-mode data. Diastolic diameter, systolic diameter, and maximal aortic radial wall velocity (RWV) (the first derivative [slope] of the aortic diameter with respect to time [dD/dt]) were determined from M-mode to assess the consistency and viscoelastic behavior of the arterial wall. Pulse diameter was calculated by subtracting diastolic from systolic aortic diameter and then normalizing to maximum systolic diameter to account for vessel size.

For morphometric analysis, the number of individual mice with ≧50%, ≧40%, or ≧30% cross-sectional diameter expansion of the suprarenal aortic segments 28 days after baseline was determined for control and LLLI mice. In addition to the individual (categorical) data, the morphometric data were also analyzed after calculating the mean cross-sectional diameter across all animals in each group (continuous data). In order to account for any normal variations in vessel sizes between animals, measurements of the suprarenal segments were also normalized to the adjacent, non-dilated, internal control segments of each animal at the level between the origins of the left and right renal arteries.

Categorical and continuous analyses were also conducted for the physiological parameters derived from M-mode data—pulse diameter normalized to systole and maximal radial wall velocity. The number of individual mice showing ≧75% reduction of pulse diameter and the number of mice showing ≧50% reduction in maximal RWV at endpoint over baseline were determined for control and LLLI mice. The means of these two parameters were calculated across all animals in each of the 4 groups.

Statistical Analysis

Categorical data were analyzed using Fisher's Exact Test with p<0.05 considered significant. For continuous data, comparisons between measurements at baseline and at the 28-day endpoint in the same mice were performed by paired, 2-tailed t-test with the Bonferroni correction for multiple comparisons. Comparisons between control and LLLI mice at baseline or at the 28-day endpoint were performed by unpaired, 2 tailed t-test with the Bonferroni correction.

Data from the M-mode measurements of one animal were excluded because of technical problems with the 28-day endpoint measurement. Data of radial wall velocity from one additional animal were excluded on basis of outlier analysis.

Results

Ultrasound measurements for each animal are presented in Table 1, shown below:

TABLE 1

| Ultrasound | | | | | | | | | | | |
|---|---|---|---|---|---|---|---|---|---|---|---|
| Measurements | | | | | | | | | | | |
| | Time point | End | | | | | | | | | |
| | Baseline | point | | | | | | | | | |
| | Ultrasound | | | | | | | | | | |
| | mode | | | | | | | | | | |
| | B-mode | M-mode | B-mode | M-mode | | | | | | | |
| GROUP | # | MSR-B | Ren-B | Dias-B | Syst-B | Veloc-B | MSR-E | Ren-E | Dias-E | Syst-E | Veloc-E |
| Control | 1 | 1.14 | 1.05 | 1.02 | 1.21 | 4.3 | 1.41 | 0.94 | 1.27 | 1.37 | 1.6 |
| Control | 2 | 1.18 | 0.94 | 1.19 | 1.31 | 4.13 | 1.51 | 0.84 | 1.41 | 1.61 | 3.31 |
| Control | 3 | 1.16 | 0.92 | 1.19 | 1.3 | 3.23 | 2.32 | 0.84 | 2.1 | 2.11 | 0.73 |
| Control | 4 | 1.16 | 0.99 | 1.15 | 1.3 | 3.91 | 1.7 | 0.94 | 1.73 | 1.79 | 1.6 |
| Control | 5 | 1.05 | 0.72 | 1.04 | 1.15 | 2.46 | 1.72 | 0.96 | 1.6 | 1.7 | 2.23 |
| Control | 6 | 1.14 | 0.86 | 1.17 | 1.32 | 2.72 | 1.69 | 0.82 | 1.54 | 1.65 | 2.95 |
| Control | 7 | 1.06 | 0.77 | 1.02 | 1.15 | 3.02 | 1.84 | 0.94 | 1.94 | 2.1 | 3.57 |
| Control | 8 | 1.11 | 0.88 | 1.05 | 1.2 | 3.77 | 1.96 | 0.91 | 1.33 | 1.35 | 1.37 |
| Control | 9 | 1.11 | 0.81 | 1.16 | 1.31 | 3.92 | 1.34 | 0.96 | 1.18 | 1.42 | 7.6* |
| Control | 10 | 1.17 | 0.79 | 1.05 | 1.19 | 3.64 | 1.81 | 1.05 | TP | TP | TP |
| Control | 11 | 1.08 | 0.76 | 0.96 | 1.11 | 4.67 | 2.36 | 1 | 2.29 | 2.34 | 0.96 |
| Control | 12 | 1 | 0.83 | 1.02 | 1.17 | 4.92 | 1.58 | 1.05 | 1.29 | 1.5 | 4.52 |
| Control | 13 | 1.22 | 0.88 | 1.09 | 1.27 | 3.5 | 1.43 | 1.05 | 1.16 | 1.34 | 3.92 |
| Control | 14 | 1.25 | 0.88 | 1.15 | 1.26 | 2.12 | 1.64 | 0.98 | 1.36 | 1.5 | 2.12 |
| Control | 15 | 1.14 | 0.87 | 1.16 | 1.25 | 4.57 | 1.53 | 1.02 | 1.67 | 1.7 | 1.53 |
| LLLI | 16 | 1.04 | 0.9 | 0.93 | 1.03 | 3.9 | 1.28 | 0.95 | 1.03 | 1.21 | 6.28 |
| LLLI | 17 | 1.05 | 0.88 | 1.02 | 1.11 | 2.99 | 1.28 | 0.91 | 1.1 | 1.3 | 4.8 |
| LLLI | 18 | 1.15 | 0.9 | 1.02 | 1.18 | 3.35 | 1.61 | 0.94 | 1.53 | 1.6 | 2.19 |
| LLLI | 19 | 1.17 | 0.96 | 1.11 | 1.28 | 2.05 | 1.05 | 0.78 | 0.87 | 1.04 | 3.14 |
| LLLI | 20 | 1.23 | 0.92 | 1.09 | 1.21 | 4.47 | 1.57 | 1.15 | 1.51 | 1.57 | 2.89 |
| LLLI | 21 | 1.04 | 0.78 | 1.04 | 1.2 | 3 | 1.26 | 1.04 | 1.07 | 1.25 | 3.72 |
| LLLI | 22 | 1 | 0.89 | 0.91 | 1.05 | 3.87 | 1.3 | 1.04 | 1.23 | 1.37 | 3.31 |
| LLLI | 23 | 1.08 | 0.96 | 1.12 | 1.27 | 4.04 | 1.19 | 1.02 | 1.06 | 1.16 | 4.29 |
| LLLI | 24 | 1.1 | 0.85 | 0.98 | 1.15 | 3.92 | 1.36 | 1.02 | 1.16 | 1.35 | 3.98 |
| LLLI | 25 | 1.19 | 0.82 | 1.04 | 1.26 | 4.56 | 1.51 | 1.13 | 1.52 | 1.61 | 5.3 |
| LLLI | 26 | 1.11 | 0.88 | 1.03 | 1.18 | 3.4 | 1.38 | 1.14 | 1.37 | 1.5 | 4.64 |
| LLLI | 27 | 1.17 | 0.82 | 1.03 | 1.24 | 4.01 | 1.32 | 1.08 | 1.37 | 1.5 | 2.69 |
| LLLI | 28 | 1.23 | 0.8 | 1.07 | 1.24 | 4.61 | 1.15 | 0.88 | 1.22 | 1.35 | 4.56 |

LLLI = Low Level Laser Irradiation,
MSR = Maximal Supra-Renal Diameter [mm],
Ren = Inter Renal (internal control) Diameter [mm],
Syst = Peak Systolic Diameter [mm],
Dias = End Diastolic Diameter [mm],
Veloc = Radial Wall Velocity [mm/sec],
B = Baseline,
E = Endpoint.
TP = Technical Problem,
*Outlier in the analysis of Radial Wall Velocity

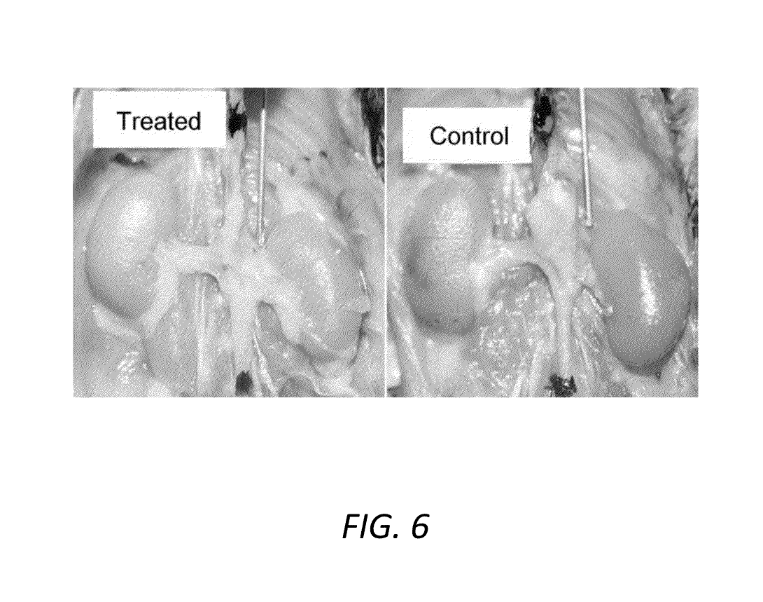

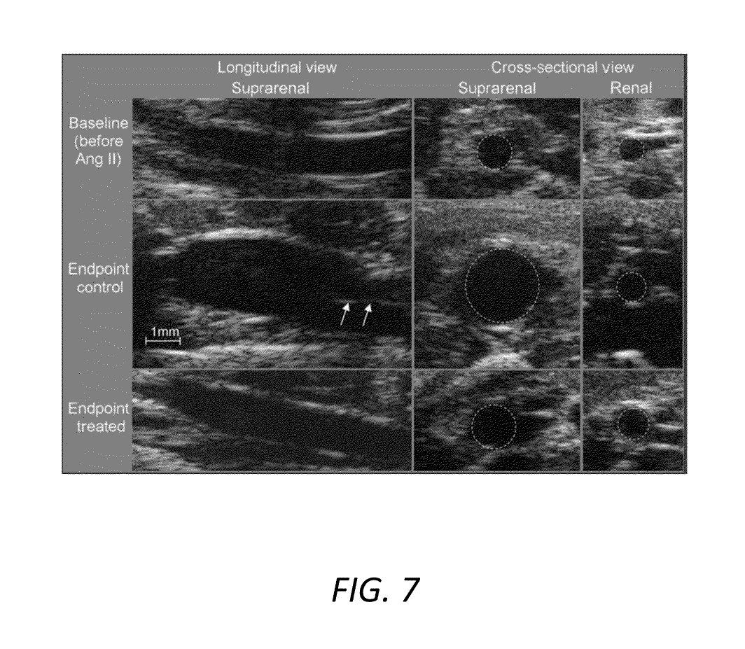

At the 4 week endpoint, 7 of 15 non-irradiated control mice had aneurysmal dilatation in the suprarenal aneurysm-prone segments of the aorta that had progressed to ≧50% expansion (maximal cross-sectional diameter [CSD]) over baseline, whereas none of the 13 low level laser irradiated mice had this degree of additional progression of dilatation (p=0.005 by Fisher's Exact Test) (Table 2, FIGS. 6,7). Likewise, when repeating this analysis for ≧40% expansion and ≧30% expansion, we found that the incidence, in mice treated with LLLI, was also significantly lower than the non-irradiated control mice (p=0.005 and p=0.003 respectively, by Fisher's Exact Test).

TABLE 2
Aneurysmal dilatation in the suprarenal aneurysm-
prone segments of the aorta over baseline.

| | >50% | >40% | >30% |
|---|---|---|---|
| Control | 7* | 9 | 11 |
| (n = 15) | | | |
| LLLI | 0 | 1 | 2 |
| (n = 13) | | | |
| P^{†} | 0.005 | 0.005 | 0.003 |

*Number of mice,
^{†}by Fisher's Exact Test

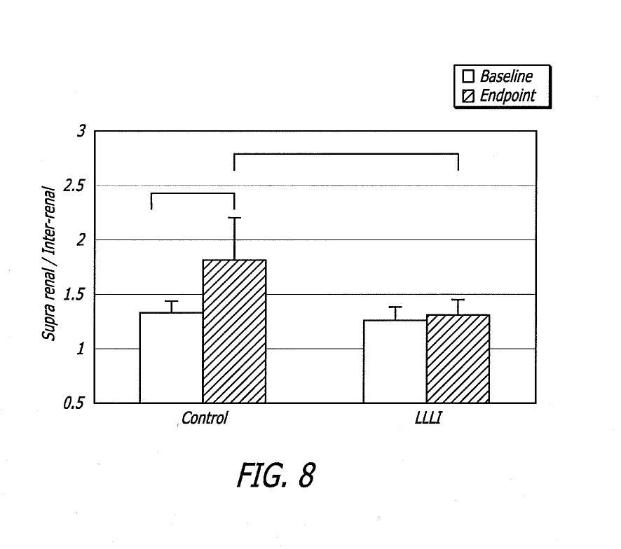

At baseline (12-13 weeks of age) there was no significant difference in the mean maximum CSD of the suprarenal segments (normalized individually to the maximal CSD of the adjacent, non-aneurysm-prone inter-renal segment) between control and LLLI treated animals (ratio of suprarenal/renal CSD, control-vs-LLLI=1.32±0.11-vs-1.29±0.13, p=0.5 by unpaired, 2-tailed t-test) (FIG. 8).

In the non-irradiated mice, the mean maximum CSD of the suprarenal aortic segments (normalized to the adjacent inter-renal aortic segments) increased significantly from baseline to 4 weeks (ratio of suprarenal/renal CSD, baseline-vs-4 weeks: 1.32±0.11-vs-1.82±0.39, p=0.0002 by paired, 2-tailed t-tests). However, in animals treated with LLLI, the maximum CSD of the suprarenal segments did not increase significantly from baseline to 4 weeks (mean of ratios of suprarenal/renal CSD, baseline vs 4 weeks: 1.29±0.13-vs-1.32±0.14, p=0.49 by paired, 2-tailed t-test).

Direct comparisons at the 4 week endpoint between the suprarenal segments of LLLI and non-irradiated animals (normalized individually to inter-renal control segments) showed a highly significant attenuating effect of LLLI on aneurysm progression in this model (mean of ratios of suprarenal/renal CSD, LLLI-vs-non-irradiated: p=0.0002 by unpaired, 2-tailed t-test).

These results show an overall 94% reduction in maximum CSD of the suprarenal aneurysm-prone segments compared to baseline in the LLLI treated mice in this model.

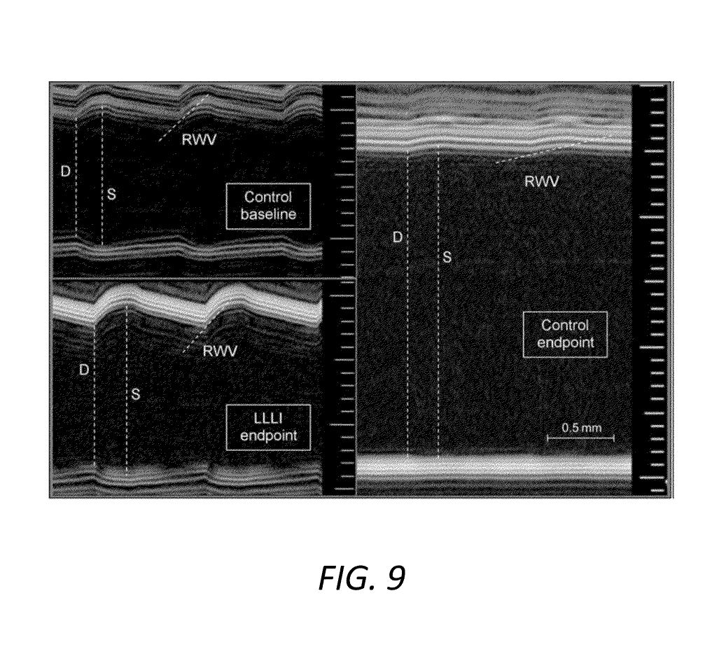

Analysis of the M-mode data (FIG. 9, Table 3) showed that the mean diastolic diameter and the mean systolic diameter were significantly higher in the control animals compared to LLLI treated animals at the 4 week endpoint. In the non-treated control mice, the mean pulse diameter (PD) (normalized to systole) of the suprarenal aneurysm-prone aortic segments 4 weeks after angiotensin infusion was significantly lower than the mean PD at baseline. However, in the LLLI treated mice, the mean PD at 4 weeks was not significantly different from baseline.

TABLE 3
M-Mode Ultrasonographic Measurements

| | | | LLLI vs | | |
|---|---|---|---|---|---|
| | | | Control at | | |
| | Control | LLLI | 4 weeks: | | |
| | Baseline | 4 weeks | Baseline | 4 weeks | p-value |
| CSD in | 1.10 ± | 1.56 ± | 1.03 ± 0.06 | 1.23 ± | 0.007 |
| Diastole [mm] | 0.08 | 0.35† | | 0.21** | |
| CSD in | 1.24 ± | 1.68 ± | 1.18 ± 0.08 | 1.37 ± | 0.005 |
| Systole [mm] | 0.07 | 0.31† | | 0.18** | |
| PD | 0.11 ± | 0.07 ± | 0.13 ± 0.03 | 0.10 ± | NS |
| | 0.02 | 0.05* | | 0.04 | |
| RWV | 3.64 ± | 2.34 ± | 3.71 ± 0.73 | 3.98 ± | 0.002 |
| [mm/sec] | 0.89 | 1.20** | | 1.15 | |

All parameters are expressed as means ± SD.

Comparisons between measurements at baseline and at the 4 week endpoint were performed by paired, 2-tailed t-test with Bonferroni correction for multiple comparisons. Comparisons between control and LLLI mice at the 4 week endpoint were performed by unpaired, 2 tailed t-test with Bonferroni correction. Baseline vs 4 weeks:

*p<0.05;
**p<0.01;
<0.001.

### CSD=Cross Sectional Diameter,

PD=pulse diameter (CSD in Systole—CSD in Diastole) normalized to CSD in systole,

### RWV=Radial Wall Velocity

When considering individual mice, at the 4 week endpoint, 4 of 14 non-irradiated control mice had more than 75% reduction in the pulse diameter (normalized to maximal diameter in systole) over baseline; whereas none of the 13 low level laser irradiated mice had this degree of reduction in PD (p=0.057 by Fisher's Exact Test).

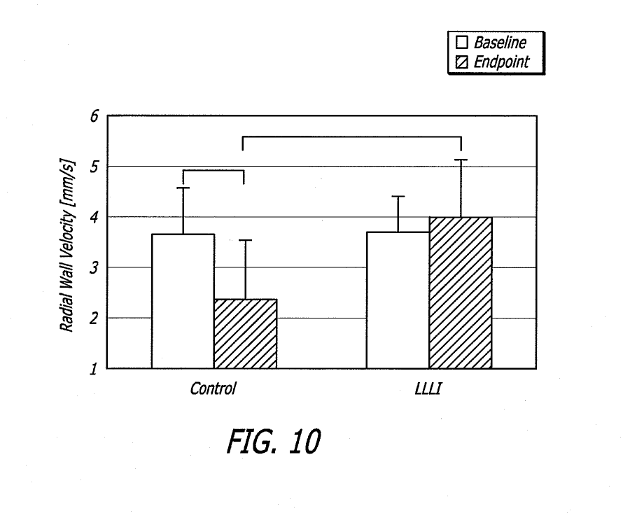

The mean maximal radial wall velocity (RWV) (FIG. 10, Table 3) of the control group was significantly lower at 4 weeks than at baseline, but no such difference in RWV was found between 4 weeks and baseline in the LLLI treated mice. However, the mean RWV at 4 weeks was significantly greater in LLL irradiated compared to the non-irradiated animals. Moreover, when considering individual mice, at the 4 week endpoint, 6 of 13 non-irradiated control mice had more than 50% reduction in the maximal RWV over baseline, whereas none of the 13 low level laser irradiated mice had this degree of reduction of RWV (p=0.007 by Fisher's Exact Test).

LLLI limited the progression of aneurysmal dilatation in the suprarenal aneurysm-prone segment of the abdominal aorta in angiotensin-II infused apolipoprotein E-deficient mice.

Whereas the higher power lasers used clinically for surgical excision and ablation convert photon energy into heat, low level lasers cause only minor temperature elevations with the usual energy densities ranging between 0.1 and 4 Joules/cm2. A variety of studies have suggested that these low energy photons are absorbed in the chromophores of the respiratory chain of the mitochondria. Studies by one of us (LG) have shown that this photon absorption apparently increases mitochondrial membrane potential. The latter is associated with an increase in the ATP energy store of the cell. This appears to represent a fundamental mechanism underlying the observed photomodulatory effects of LLLI.

Abdominal aortic aneurysm (AAA) is present in 6-10% of the population over the age of 65. Current forms of treatment are based on either open surgical repair with grafts or endovascular repair by large membrane-covered stents. Both techniques have major side effects with potentially life-threatening consequences emphasizing the importance of developing alternative therapeutic strategies, such as that presented in the current study, that target pathogenetic mechanisms of progression and rupture.

The angiotensin II-infused apolipoprotein E-deficient mouse model, developed by Daugherty and colleagues, shows important similarities to human AAA pathology.

These include degradation of the elastic tissue associated with marked inflammatory cell infiltration and disruption of the musculo-elastic lamellar structure of the media including medial dissection. Similar changes have been detected in the elastase model of aneurysm and in the periarterial calcium chloride model developed in this laboratory. In the mouse angiotensin infusion model, these histological changes are usually found before the development of more advanced proliferative atherosclerotic lesions at these sites. However, AAA in humans, most commonly found in the infrarenal position, is usually diagnosed in a vessel that already has severe atherosclerotic changes in the wall. Nonetheless, marked inflammatory cell infiltration associated with disruption of the musculo-elastic architecture of the media is a major underlying histopathological process common to AAA in both animal models and man.

Measurements obtained by femoral artery catheter and the tail cuff method confirmed that angiotensin II infusion did not increase blood pressure in this model, and hence the suprarenal aneurysms, and the associated wall changes are considered to occur independently of this parameter.

High frequency ultrasonography (0.01 mm resolution) used in the current study was designed specifically for non-invasive microimaging in mice. Previous studies in the same mouse model showed a high correlation with direct post-mortem measurements of the outer diameter of the aorta obtained by digital photography and a very high correlation with morphometric measurements of H&E stained histological sections (r=0.99). The measurements were also shown to have relatively small intra- and inter-individual variance. These studies support the accuracy and reproducibility of non-invasive high resolution ultrasound monitoring of the dimensions of AAA in living mice including the effects of investigative manipulations and treatment regimens over time. Histology will be necessary to study effects of LLLI on cell and tissue morphology and pathobio logy in this mouse model. However, the current study was designed to establish the effect of LLLI on aneurysm progression in vivo without the need for corrections of measurements for tissue shrinkage, embedding misalignment, or other perfusion fixation and preparation-related deformations of the arterial wall which confound accurate evaluation of wall shape by histopathology.

We have shown that, in addition to a marked reduction in the mean maximum diameter of the supra-renal aortic segments of LLLI treated versus non-treated control aortas, the number of individual animals that developed aneurysmatic dilatation with greater than 50% increase in maximum CSD over baseline was significantly lower in the laser treated group. This degree of maximum diameter increase over baseline (or adjacent control segment) has been considered to be the accepted increment required for classification as a significant aneurysmal dilatation.

Absolute measurements of cross-sectional diameter of the aorta can be considered sufficient for assessment of changes in aneurysmal dilatation from baseline to endpoint provided that both measurements are made in the same animal. However, when comparing the degree of aneurysmal dilatation in treated versus non-treated animals, use of absolute measurements alone fails to consider possible anatomical differences in vessel wall size between animals. Thus, in the current study, comparisons between treated and non-treated animals were be performed after normalizing the diameter measurement of the suprarenal aneurysm-prone segment to that of the adjacent, non-aneurysm-prone, non-dilated, internal control, inter-renal segment whose aortic diameter has been shown not to change over 28 days in this model.

Analysis of M-mode data showed that the mean pulse diameter of the suprarenal aneurysmatic segment of non-treated control mice decreased from baseline to the 4 week endpoint. However, this was less pronounced in the LLLI treated animals, and the number of individual mice with significant reduction in PD was somewhat less (p=0.057). Reduced PD is a reduction in the difference between the arterial wall diameter of the affected segment in systole and that in diastole. This occurs in situations of arterial wall weakening and/or reduced wall elasticity leading to reduced contractility such as that associated with aneurysmal dilatation. This is also consistent with data accumulated from a variety of studies including from our laboratory which have identified marked changes in matrix protein expression, secretion, and degradation in this and other models of aneurysm formation.

From M-mode data we also found a significantly lower mean maximal radial wall velocity in control mice at the 4 week endpoint than that measured in the LLLI treated mice, and a significantly greater number of control mice with more than 50% reduction in this parameter as compared to the LLL treated mice. Maximal RWV is the first derivative, or slope, of the aortic diameter over time. Reduction in the maximal RWV occurs with a decrease in the speed of the wall motility during the cardiac cycle. This phenomenon presents as changes in the echogenic texture of the vessel wall. Such changes are seen in cases of increased inflammatory cell infiltration that accompany changes in visco-elastic behavior of the arterial wall. That mice treated with LLLI in the current study showed significantly less reduction in RWV than non-treated controls is consistent with our in vitro findings of the effects of this modality on expression and secretion of inflammatory chemokines and cytokines, the effects on cell proliferation and matrix protein secretion, and the known empiric effects on a variety of clinical entities where inflammation is a major pathogenetic substrate.

In conclusion, we have shown that LLLI significantly attenuates aneurysm formation in the angiotensin II-infused apolipoprotein E-deficient mouse. These studies, when considered together with previously reported in vitro studies, appear to provide strong support for initiation of studies in large animals as the next step toward testing the applicability of this technology to the human interventional setting.

It will be apparent from the foregoing that while particular forms of the invention have been illustrated and described, various modifications can be made without departing from the spirit and scope of the invention. Accordingly, it is not intended that the invention be limited.

What is claimed is:

We claim:

1. An implantable apparatus for internal treatment of body cavities and damaged vessels using electromagnetic radiation said apparatus comprising:
an implantable light source operationally connected to an implantable illuminating surface, wherein the implantable light source and implantable illuminating surface are:
i. of materials, which are compatible with implantation in a human subject;
ii. possessed of an expanded, deployed configuration and compact, undeployed configuration suitable for laparoscopic insertion within a body cavity for application, when deployed, to an outer, periadventitial surface of an internal vessel or surface of an organ; and
a control unit, which control unit regulates the activation of said light source and is optionally located remotely from the implantable light source and illuminating surface.

2. The implantable apparatus of claim 1, wherein said implantable light source comprises at least one light emitting diode (LED).

3. The implantable apparatus of claim 1, wherein said apparatus further comprises a drug or biologic eluting material/matrix onto which or within which said implantable light source, operationally connected to an implantable illuminating surface, and, optionally said control unit, are located.

4. The implantable apparatus of claim 1, wherein said implantable light source and implantable illuminating surface are modular, electrically coupled and can be interconnected, whereby the dimensions of each of said implantable light source and implantable illuminating surface may be selected to suit a particular application.

5. The implantable apparatus of claim 1, wherein said implantable light source and implantable illuminating surface are selected to be of a size that is suitable to a particular application.

6. The implantable apparatus of claim 1, wherein electromagnetic radiation applied is of wavelengths specifically between 500-900 nanometers.

7. A method for the internal treatment of body cavities and damaged internal vessels using electromagnetic radiation, comprising providing an implantable apparatus of claim 1 applied to a tissue surface adjacent to the surface of tissue to be treated and irradiating the latter tissue surface.

8. The method of claim 7, wherein said method comprises introducing said implantable apparatus into a body cavity by a laparoscopic or other endoscopic procedure.

9. The method of claim 7, wherein said method comprises introducing said implantable apparatus into a body cavity by an endovascular placement system.

10. The method of claim 7, wherein said step of introducing said implantable apparatus into a body cavity by an endovascular placement system comprises mounting said implantable apparatus on an exterior surface of an endovascular placement system.

11. The method of claim 10, wherein said endovascular placement system comprises a stent.

12. The method of claim 7, further comprising the step of remotely controlling a remote electrical source by RF coupling from an external source.

13. The method of claim 7, further comprising varying a radiation wavelength.

14. The method of claim 7, further comprising varying a radiation frequency.

15. The method of claim 7 further comprising varying the radiation energy level.

16. The method of claim 7, further comprising varying a surface treatment by time phasing of the energy from the light source.

17. An implantable apparatus for internal treatment of body cavities and damaged internal vessels using electromagnetic radiation, comprising: a matrix attachable to a tissue surface to be treated; a plurality of electromagnetic energy sources mounted to said matrix; and a remote electrical source in electrical communication with said plurality of electromagnetic energy sources for activation and control of energy emitted by said electromagnetic energy sources.

18. The implantable apparatus of claim 17, wherein said matrix further comprises a drug or biologic eluting material.

19. A method for the internal treatment of body cavities and damaged vessels using electromagnetic radiation/energy, comprising an implantable apparatus of claim 17 onto or proximal to a tissue surface to be treated, and irradiating the tissue surface.

20. The method of claim 19, wherein said method comprises introducing said implantable apparatus into a body cavity by a laparoscopic or other endoscopic procedure.

21. The method of claim 19, wherein said method comprises introducing said implantable apparatus into a body cavity by an endovascular placement system.

22. The method of claim 19, further comprising the step of remotely controlling a remote electrical source by an RF coupling from an external source.

23. The method of claim 19, further comprising varying a radiation wavelength.

24. The method of claim 19, further comprising varying a radiation frequency.

25. The method of claim 19, further comprising varying the energy level.

26. The method of claim 19, further comprising varying a surface treatment by time phasing of the energy from the light source.
